(19) United States
(12) Patent Application Publication
(10) Pub. No.: US 2012/0197003 A1
(43) Pub. Date: Aug. 2, 2012

(54) Modified oligopeptides for the treatment of pancreatitis, stomach ulcers, and other hyperenzymemias based on enzyme peptide inhibitors and methods for obtaining them

(76) Inventors: Artur Martynov, Kharkov (UA); Boris S. Farber, Brooklyn, NY (US); Sonya Sophya Farber, New York, NY (US)

(21) Appl. No.: 12/931,461

(22) Filed: Feb. 1, 2011

(51) Int. Cl. C07K 2/00; C12P 21/00

(52) U.S. Cl. 530/300; 435/68.1

(57) ABSTRACT

Modified oligopeptides for the treatment of pancreatitis, stomach ulcers, and other hyperenzymemias based on an enzyme peptide inhibitor where in the capacity of the main active ingredient, a mixture (assembly) of chemically modified oligopeptides are used that are products of autological hydrolysis of the same proteolytic enzymes of trypsin, chymotrypsin, and papain, with changes of their molecular charges to the opposite through acylation with succinic anhydride and alkylation by monochloracetic acid.

### FIELD OF APPLICATION

This invention may be used for the creation of peroral and parenteral medicines in the pharmaceutical and medical fields for the treatment of pancreatitis, stomach ulcers, and other hyperenzymemias.

### SUMMARY OF THE INVENTION

Modified oligopeptides for the treatment of pancreatitis, stomach ulcers, and other hyperenzymemias based on an enzyme peptide inhibitor where in the capacity of the main active ingredient, a mixture (assembly) of chemically modified oligopeptides are used that are products of autological hydrolysis of the same proteolytic enzymes of trypsin, chymotrypsin, and papain, with changes of their molecular charges to the opposite through acylation with succinic anhydride and alkylation by monochloracetic acid.

### TECHNICAL RESULT

The mixture (assembly) of modified oligopeptides obtained quickly stops the inflammatory process caused by excess secretions of proteolytic enzymes. The technology of the proposed drug is simple, economically accessible, ecologically harmless, and waste-free, and contains three stages. Due to the effect of selective complementary hybridization, the peptides achieved interact only with their enzyme ancestors, thereby inhibiting their function. The low molecular weight of the peptides obtained and their immunity to hydrolytic enzymes allows them to easily penetrate through biological membranes, be absorbed from the intestine, and collect in areas with high concentrations of enzyme targets. These properties permit the peptides being patented to be used to obtain, among other things, peroral medicinal formulae for the treatment of chronic pancreatitis and stomach ulcers.

Modified Oligopeptides for the Treatment of Pancreatitis, Stomach Ulcers, and Other Hyperenzymemias Based on Enzyme Peptide Inhibitors and Methods for Obtaining Them

### TECHNICAL FIELD

This invention is related to medicine—specifically, to gastroenterology—and is intended for the treatment of pancreatic, stomach, and intestinal pathology in humans.

### Modern Level of Technology

### The Use of Enzymes and Their Inhibitors in Medicine and Pharmacology

The use of enzymes and other physiologically active substances (FAS) of a protein nature in therapy has a long tradition. Modern medicine is using highly cleaned FAS drugs of a protein nature in varied branches of clinical medicine in the capacity of promising medicinal therapies as a result of their exclusively high levels of activity and specificity. At present, the following major areas of enzyme therapy have been noted: 1) elimination of an enzyme deficit with the goal of compensating for genetic or acquired functional deficiencies; 2) removal of nonviable, denatured structures and cell and tissue fragments; 3) lysis of clots; 4) complex therapy of malignant neoplasms; 5) detoxification of the body. All these aspects have been more or less widely considered throughout the past ten years, both in original articles and in summary works [¹, ², ³]. The use of enzymes in replacement therapy has been practiced for a long time; the necessary increase in the level of enzymes in the intracellular and digestive fluids is attained relatively easily. For treatment of digestive ailments, many drugs are proposed that contain a mixture of enzymes of animal and vegetable origin, which break down proteins, fats, carbohydrates, and other components. We do not have the opportunity to encompass the broad field of digestive and metabolic ailments, examine various indications for replacement therapy, and list the drugs that exist. These issues have been brought to light in detail [2]; we will present just a few examples. When endocrinal deficits of the gallbladder arise, special meaning is given to the introduction of pancreatic enzymes; to prevent unpleasant symptoms from arising, it is recommended that drugs be taken that contain, in addition, bile salts, acids, and cellulases. Hydrolytic enzymes (most often papain, bromelain, and pancreatic enzymes in combination with cellulases) have been used with success over recent years to dissolve non-fatty gallstones. The key factor that affects the effectiveness of oral enzyme therapy is the inactivation of pancreatic enzymes under the influence of the digestive juices. With the goal of raising the local pH of the stomach to 4.0, combining protease with bicarbonate of soda or aluminum hydroxide is recommended. Another approach that permits the lessening of the harmful effect of the stomach contents on protease is obtaining enzyme drugs with a coating that dissolves in the intestine. The treatment of genetic enzymopathies, of which more than 150 have been described over the last twenty years, is an important problem of replacement therapy. Inherited diseases such as glycogenoses, lipidoses, mucopolysaccharidoses, and other liposomal diseases, have seen treatment attempts with the intravenous introduction of the corresponding native enzymes taken from human biological fluids and tissues [⁴]. However, satisfactory results have not been obtained, especially for liposomal diseases characterized by a pathological aggregation of substrate in nerve cells. The reason for the lack of success is the quick introduction of native enzymes from the blood flow and their pick-up by the liver, and generally the insurmountability of the blood-brain barrier and the impossibility of the enzyme's penetration to neuronal liposomes. The clinical application of enzymes (proteases, collagenases, and hyaluronidases) for the treatment of various pyoinflammatory processes is based on their necrolytic, mycolytic, and antiedemic activities and on their ability to decrease the antibiotic resistance of microbail flora. Proteases of animal and bacterial origin are used in surgery in the treatment of septic illnesses of the soft tissues, bones (in osteomyelitis and pyarthrosis), and lungs and pleurae (in tuberculosis). One of the most important areas of proteinase application is heat burns. Local enzyme therapy of deep burns reduces lethality in the toxemic period, speeds up the cleaning and regenerative process, and in the majority of cases prevents the need for skin grafts. Enzyme therapy is effective in traumatology and orthopedics; it facilitates a reduction in treatment periods for breaks, dislocations, sprains, and muscle ruptures. Hyaluronidases are used fairly widely in orthopedic clinics for the treatment of scars of various origins, contractures, and so on. Enzyme therapy is used quite actively in otolaryngology for the treatment of diphtheria, tonsillitis, laryngitis, and otitis. In dental practices, protease is very promising in purulent surgery in the maxillofacial area and in the treatment of the tissues of the periodontium. Thus the data quoted indicate that the areas of application of proteinase for the treatment of inflammatory reactions are practically limitless. In medical enzymology, there is hardly a problem that has been studied as widely and intensively as the problem of clot lysis. Severe vesicular thromboembolisms remain the main reason for the illness and death of the elderly. We will not spend time on the examination of modern ideas on blood clotting mechanisms and thrombolysis; the literature in that field is extensive and accessible [⁵]. We will simply say that the formation and lysis of blood clots are a quite complex cascade of proteolytic processes, in which key positions are held by plasmin, thrombin, and fibrin. Two basic approaches to thrombolytic therapy are possible: 1) the use of activators that change plasminogen to plasmin, such as streptokinase and urokinase; 2) the use of proteases that have a direct fibrinolytic action, such as plasmin itself, trypsin, and chymotrypsin. In many cases, a combination of enzymatic and anti-coagulant therapy is recommended. In recent years, trypsin-type enzymes taken from snake venom have seemed promising, in the presence of which fibrin clots and the mechanically less strong and therefore more sensitive to lysis currant jelly clots form [1]. The use of streptokinase and urokinase in the treatment of myocardial infarction and pulmonary embolism has led to fast, stable improvement; however, after parenteral introduction of streptokinase, allergy symptoms did arise [1]. Proteolytic enzymes of various origins have turned out to be effective in different blood vessel diseases such as arterial thrombosis (peripheral and cerebral) and surface and deep thrombophlebitis [1]. The mechanism of action of trypsin and chymotrypsin in cases of pathological intravascular clot formation is determined by their anti-inflammatory and antiedemic actions and their ability to activate the blood's anti-clotting system, which is reflected in the increase in fibrinolytic activity of the plasma while the clotting system indicators show practically no change. In connection with the examination of various examples of the medical application of proteolytic enzymes, it is appropriate to bring up the widespread use of their protein inhibitors. Indications for their use are: 1) a genetic deficit of proteinase inhibitors, including antitrypsin; 2) pathological activation of proteolytic processes connected with induced non-physiological protease release or activity of exogenic enzymes in the presence of microbial pathology.

Natural protease inhibitors taken from the pancreatic and partoid glands and the lungs of large horned stock have been the most widespread. These inhibitors effectively inhibit plasmin, plasmin activators, blood clot activators, kininogenase, trypsin, chymotrypsin, and tissue and leukocyte proteinases [⁶]. Polyvalent protease inhibitors are used in clinical practice in the presence of fibrinolytic blood loss arising after surgical intervention, as they slow the formation of thromboplastins in combination with plasmin activity. The protease inhibitors mentioned are used in the treatment of sepsis, bacterial (endotoxic) shock, allergic reactions, severe and post-operative pancreatitis, mechanical and thermal injuries, and arthroso-arthritis [⁷]. When a myocardial infarction takes place, proteolysis inhibitors provide anti-ischemic activity, decreasing the necrotic zone and improving collateral circulation. In recent years, the effectiveness of the anti-viral activity of proteolysis inhibitors has been discovered in clinics and in experiments. However, for the expansion of the area of application of enzyme inhibitors, inhibitors must be found that have a wider spectrum of activity: in part, those that are capable of slowing neutral, alkaline, and liposomal proteinases. The use of enzymes in oncology occupies a special place. Attempts have been made to use proteases, nucleases, and mycopolysaccharides with the goal of direct lysing action on cancer cells. Their introduction into a tumor has a marked effect; parenteral introduction has a weaker effect due to the presence of protease inhibitors in the blood serum of cancer patients. The field of enzyme therapy on neoplasms is currently considered more promising; this is based on the recognition of the peculiarities in the metabolism of cancer cells and the proposed use of enzymes that would decrease the concentration of metabolites and nutrient substances in the blood flow and the tumor cells. The best-known cytostatic enzyme substance of this type is L-asparaginase. This enzyme became a standard drug for the treatment of lymphoblast leukosomes [1]. However, toxicity, especially allergic reactions, and the fast development of resistance to the enzyme restrict its application. The discovery of the specific effect of L-asparaginase on cancer cells caused other enzymes that break down replaceable and nonreplaceable amino acids to be studied. Anti-tumor activity was discovered in glutaminase, arginase, and arginine decarboxylase [1], as well as in enzymes that lower levels of individual nonreplaceable amino acids such as a-hydroxilase, lysine oxidase, histidase, and phenylalanine ammonia lysase [1]. The activity of these enzymes increases in the presence of antimetabolites of amino acids through inhibition of biosynthetic pathways and a change in the concentration of amino acids. In cancer therapy, the use of enzymes that take part in the catabolism of folic acid, folate coenzymes, or folate antagonists is described. Folate coenzymes are necessary for the biosynthesis of purines and thymine. The most promising for the degradation of folic acid is the use of carboxypeptidase G. Notwithstanding its noticeable immunogenicity, the enzyme in the experiment has anti-tumor activity; moreover, it is effective for the prevention of toxicity in the well-known inhibitor dihydrofolate reductase methotrexate in the treatment of leukemia and brain cancer [1]. We therefore see that drugs that are natural, physiologically active protein compounds (enzymes, their inhibitors and activators, hormones) have acquired a worthy place among the substances used in practical medicine. Unfortunately, everyday clinical use of enzymes is limited both by economic factors (their high cost and low availability) and by their fast inactivation in conditions inside the body and the various side effects they cause as foreign proteins (antigenicity, allergy, toxicity, and so on). To a significant extent, the obstacles mentioned may be overcome through the use of enzymes in a stabilized, immobilized form, especially because through the efforts of enzymology engineering, a number of methods of covalent and non-covalent fixation of enzymes on various insoluble and soluble carriers have been developed as of today. At present, it is customary to set out two principal approaches to the obtainment of immobilized therapeutic enzymes. In various cases of systemic attacks when the presence of a therapeutic enzyme is necessary in various organs and tissues, it is useful to somehow use stabilized, water-soluble drugs made of immobilized enzymes that have high levels of stability in physical conditions and slow elimination speeds from the body. Various enzyme-containing artificial cells such as microcapsules, erythrocyte ghosts, or liposomes also belong to this group. On the other hand, for the treatment of local damage when the presence of the enzyme is required only at the location of the damage, it is useful to create biocompatible, enzyme-containing (biodegradable or simply temporarily implanted) polymer particles that can be localized in a given place and remain there for a set time, constantly releasing a therapeutic enzyme (which is preferably additionally stabilized) into the environment. Other cases could be the use of immobilized enzymes in equipment for the extracorporal perfusion of the artificial kidney type, in bandaging and drainage materials to speed wound and burn healing, and for the modification of the internal surfaces of prosthetic blood vessels with the goal of decreasing blood clot formation. The stabilization of therapeutic enzymes in many cases may be done without use of polymer carriers, simply through goal-oriented chemical modification of the protein globule with low-molecular reagents and through implanting into the globule intramolecular brackets made of bi-functional reagents that make denaturing developments in the protein molecule difficult [⁸]. This approach is especially important if the therapeutic enzyme must, in order to fulfill its function, interact with the cell membrane receptor (for example, a thrombin-thrombocyte pair) or penetrate into the cell (enzymes used for the treatment of many genetic enzymatic deficiencies of the liver), and the presence of a polymer carrier may sharply decrease the effectiveness of the enzyme drug. In many cases, intermolecular binding of ferments with bi-functional reagents of the glutaric aldehyde type is used, which may also be seen as the immobilization of one molecule of enzyme on another. In many cases, this modification to the enzyme leads to an increase in its stability and effectiveness—for example, a-galactosidase, used to treat Fabry's disease, has been stabilized in this way. The binding of enzymes with other proteins also has a marked effect: conjugates of uricase or hemoglobin with albumin are capable of circulating in the intravenous blood flow in active condition for many times longer than their corresponding native proteins. However, it should be borne in mind that large protein formations with many different areas interacting with receptors of various cells may in certain cases be picked up, for example, by liver cells with increased intensity. The most widespread method of obtaining soluble stabilized enzyme drugs is their modification with soluble polymers [⁹]. The most convenient carriers are polysaccharides, including dextrans, due to their high level of biocompatibility [¹⁰]. Certain nontoxic and non-immunogenic synthetic polymers—for example, reactive derivatives of polyvinylpyrrolidone, polyethylene glycol, polyvinyl alcohol, and so on—may also be used to immobilize therapeutic enzymes. Certain perspectives are opened up by the use of natural bonds as carriers that have, in and of themselves, beneficial physiological activity or are capable of strengthening the activity of the enzyme they are bonded to. Thus the anticoagulant heparin can be used to immobilize thrombolytic enzymes [¹¹]. When using these drugs, it must be kept in mind that their molecular mass must not exceed 80-100,000, since otherwise their removal from the body after they fulfill their function will be difficult, and the buildup of polymer carriers in the body may cause unpredictable complications. With this goal, in recent years the development of reactive synthetic polymers that contain bonds capable of biodegradation has been going on. This means that they will not collect in the body even if there is a large molecular mass [¹²]. Bonding therapeutic enzymes to soluble carriers is done through the traditional methods of immobilization, which are developed in detail. The polymer carrier in and of itself may be either bioindestructible (amide, ether) or biodestructible (urethane, schiff base, etc.). The principal effects achieved due to the bonding of a therapeutic enzyme or another protein bond (for example, a hormone such as insulin) are the following: an increase in the conformative stability and resistance to the activity of endogenous proteases; an increase in the circulation time in the blood due to a rise in the molecular mass of the conjugate and the slowing of its elimination; the opportunity to regulate the immune response of the body to the protein drug; finally, the chance to obtain drugs with complex therapeutic activity. The possibility of regulating the body's immune response to the introduction of a therapeutic enzyme is extremely important, as many promising enzyme drugs cannot be used due to the fact that they provoke reactions in the body as foreign proteins. At the same time, the immobilization of these enzymes on natural or synthetic polymers, for example on polysaccharides or polyethylene glycol [¹³, ¹⁴], sharply decreases the immunological and allergic reactions of the body, apparently due to the carrier matrix's creation of steric hindrances to antigen-antibody interaction. On the other hand, it has been proven that certain synthetic polyelectrolytes strengthen the immune response to the protein bonds connected to them, which opens up the possibility of the creation of a new type of synthetic vaccine. As has already been noted, proteolytic enzymes are the most widely applied in clinical practice. It is therefore unsurprising that a large number of studies have been dedicated to obtaining their immobilized derivatives. It has been demonstrated that conjugates of protease with soluble carriers—subtilisin, trypsin, chymotrypsin, terrilitin, and others—may preserve in nearly unchanged form their specific activity in a low-molecular substrate while at the same time having, in comparison to their native predecessors, high stability in relation to various denaturers through the effect of increased lifetime in the blood flow. Drugs of this type have already found clinical application: the USSR was the first in the world to have implemented in practice streptokinase immobilized on a polysaccharide carrier successfully used in the treatment of various thrombolytic diseases without causing side effects. Among other hydrolytic enzymes, liposomal p-D-N-acetyl glucose amidinase should be mentioned, which is used to treat Tay-Sachs disease, and for which it was proven that its immobilization on polyvinylpyrrolidone [¹⁵] leads to stabilization in relation to exogenous proteinase and to an increase in the time of its activity in the blood flow of experimental animals. Potentially anti-tumor drugs, such as nucleases, which also have anti-viral activity, sharply improve their properties—the stability of their biological activity—when they are bonded with soluble aminoderivatives of dextran by the azocoupling method. Soluble drugs made of immobilized enzymes may be used not only for intravenous introduction; carboxylpeptidase G and arginase modified with dextran [¹⁶], when parenterally introduced in mice with implanted mastocytoma, have the ability to create a higher and longer-acting concentration of the agent in the blood flow than do native enzymes. Many more examples of this type could be listed, but they would all lead to one thing: the immobilization of enzymes on soluble polymer carriers allows us to obtain more stable, active, and safe therapeutic drugs. Moreover, the methods developed for the immobilization of enzymes may be successfully transferred to other drugs of a protein nature: various physiologically active polypeptides of the type of the pancreatic inhibitor trypsin and, especially importantly, the hormone insulin. Another promising method of applying modified forms of enzymes for treatment is the creation of various types of artificial cells. Historically, the first approach to this problem was the microencapsulation of enzymes: that is, their inclusion in polymer microcapsules that facilitated reliable containment and protection of the enzyme and the free penetration of relatively low-molecular substrates and products of the enzymatic reaction. The main advantages to microencapsulation are that the microcapsule excludes contact between the enzyme and biological fluids; a relatively high concentration of the enzyme may be included in the microcapsule that could not have reached the blood flow if the enzyme had been used in native form; various enzymes can be included in the microcapsule at the same time; enzymes in microcapsules may be additionally stabilized by intra- or intercellular binding or modification by soluble polymers. Moreover, considering that there are now approaches to obtaining microcapsules not only from synthetic polymers (polyamides, polyurethanes, and so on), but also from natural ones or their analogues (polylactic acid and so on), that is, the problem of the use of microcapsule covering material in the body is being removed, these kinds of enzyme drugs are now seeming more promising. It should be emphasized, however, that their use is limited to cases in which the therapeutic enzyme must act on a soluble substrate of relatively low molecular weight. The first successful experiments in the use of microencapsulated enzymes in animals were conducted with the use of urease (to decrease the quantity of urea in the blood), catalase (to treat animals with catalase deficiencies), and asparaginase (to reduce the growth of asparagin-dependent tumors). The first example of the use of microencapsulated enzymes in clinical practice is the use described in a work [26] of microencapsulated catalase for the treatment of wounds to the oral cavity in humans that had formed in cases of Takahara's disease as the result of the aggregation of hydrogen peroxide emitted by bacteria. The second-oldest method of creating artificial cells is the inclusion of enzymes in liposomes: artificial phospholipid microbeads. The enzymes included in the liposomes are also safeguarded from inactivating activities from the outside environment, while the liposomes themselves, as they are made up of natural bonds are fully used in the body. Unlike microcapsules, however, liposomes have the unique ability to deliver the drugs they contain inside the cells with which they interact through the mechanisms of conjugation or endocytosis. If the group of diseases mentioned above that are connected with a deficit of intracellular liposomal enzymes is taken into account, it is difficult to overemphasize that fact. The vast majority of enzymes used in clinics have been included in liposomes of a given effect; data of this kind is exhaustively described in monographs [¹⁷]. Data on the first successful experiment with liposomally encapsulated enzymes in a clinical setting are also presented therein: the glycocerebrosidase included in the liposomes (unlike the native enzyme, which is not capable of penetrating into a cell) turned out to be quite effective in the treatment of a patient with Gaucher's disease, connected with the disruption of the normal metabolism of glycocerebrosidases that collect in the cells of the reticulo-endothelial system. Amyloglucosidase included in liposomes have been successfully used in the treatment of a patient with type II glycogenosis. If account is taken of the fact that the cells of the reticulo-endothelial system, including the liver, are natural targets for liposomes, it is clear that enzymes included in liposomes may be quite effective in the treatment of various enzymatic deficits of the liver. Liposomes, whose surfaces lend themselves to various modifications, may be used as containers for the directed transportation of medicines, due to which it is now accepted that the medicine introduced is capable of collecting specifically in a damaged zone. In many experimental works, it has been demonstrated that liposomes whose internal layers include various drugs and to whose outer membrane a vector compound is chemically bonded are capable of recognizing and specifically bonding with damaged zones (for example, an antibody to a specific component of a target zone) and truly selectively concentrates in a set place. This approach can expand the arsenal of methods of enzymatic therapy even further. Cell and blood coverings such as membranes or erythrocyte ghosts, can be used as containers for enzymes [¹⁸]. Through the partial hemolysis of erythrocytes and their subsequent filling with the desired contents with full recovery of membrane entirety, various enzymes have been included in them; it has been demonstrated that the included enzymes significantly increase the time of their presence in the blood flow in active condition (experiments were conducted on mice and monkeys). An attempt to use glucocerebrosidase included in erythrocytes for the treatment of a patient with Gaucher disease has also been described [¹⁹]. For the treatment of local, rather than systemic, diseases, clearly drugs using immobilized enzymes must be created that can be localized in the required area and are capable of releasing an active enzyme into the surrounding environment at a set speed, thereby creating a local depot of enzyme drug in the body. As a result, a high local concentration of the drug may be attained, while its total dosage will be insignificant in comparison with administration of native enzymes in solution form. These drugs may be obtained by several methods. In the first place, they may be included in a solution of synthetic biocompatible polymer, from which tablets or granules are then formed that are designed for implantation and capable of slowly releasing the protein drug included in them (experiments have been conducted with trypsin, lysozyme, alkaline phosphatase, catalase, and others) [²⁰]. The speed of the enzyme's release may be regulated by the density of the polymer matrix. While in the polymer matrix, the enzyme is protected from the effects of the aggressive biological environment, as a result of which these systems may function for a long time. In that case, the unsolved problem remains the fate of the polymer implant after the full release of the agent. In the second place, enzymes may be included in the process of formation in the structure of various fibers and films applicable in the capacity of suturing, bandaging, or drainage materials. Due to the principle of gradual expulsion, the materials with the included proteolytic enzymes have high-level and long-term cleansing, draining, and thrombolytic activity, are capable of working without being changed for a long period of time, and can speed up the healing process by several times in comparison with traditional therapy methods. Drugs made from immobilized enzymes for local application may be obtained both on the basis of insoluble polymers (here, the enzyme fulfills its function while connected with the carrier and then is mechanically removed from the damage center) and on the basis of biodegradable carriers, where the speed of the elimination of the enzyme into the environment is determined by the speed of the breakdown of the carrier, with which the enzyme is covalently bonded. With the use of microgranules of cross-linked dextrin—Sephadex—drugs made of thrombolytic enzymes were created with a set speed of biodegradation; it was demonstrated that fibrolysine, streptokinase, or urokinase immobilized in this way can be used for local deposition in clot therapy [²¹]. In this case, effective thrombolytic therapy in experiments on animals achieved, with the use of general doses of enzymes that were half the amount of those used in traditional therapy methods. This kind of drug can be deposited in practically any organ and is absolutely promising for clinical application. Another large field of enzyme application in medicine that has opened up only as a result of the development of methods for immobilizing them is the use of immobilized enzymes in various extracorporal apparatuses for the perfusional purification of various biological fluids. This general approach is often used in the creation of enzyme reactants that can be used as clot-safe blood vessel prostheses. The main advantages of the reactants based on immobilized enzymes are the following: the opportunity to avoid direct contact of the body with a foreign protein, thus decreasing undesirable reactions to that protein;
the opportunity to use the reactant many times; the opportunity to conduct long-term treatment. The shortcomings of these reactants are the incompletely addressed problems of clot formation or the sorption of blood proteins on foreign surfaces. Extracorporal perfusion with the use of enzyme reactors is already used fairly widely. The use of asparaginase is described in the most detail; it is included in varied reactors and used for the treatment of asparagin-dependent solid tumors. In animal and human experiments, it has been demonstrated that the inclusion of immobilized asparaginase in the circulatory system allows the quick and quantitative removal of asparagine from the blood. Similar systems with immobilized uricase or L-phenylalanine-ammonia-lyase may be used for the general detoxification of the body due to their ability to decrease the level of uric acid in the blood of animals with hyperuricemia or the level of phenalalinine in cases of phenylketonuria. In conclusion, it should be noted that the possibilities and perspectives of the use in medicine of enzymes and their inhibitors in their immobilized state are much wider than have been obtained at present. Without a doubt, it is down this road that the creation of new and highly effective treatment methods awaits the field of medicine.

New components that have anti-trypsin activity are known. These components are aldehyde analogues of peptides that specifically and quickly inhibit pancreatic trypsin in mammals. The components are useful for the prevention and treatment of tissue breakdowns associated with pancreatitis [²²]. At the basis of the patent lies the method of aldopeptide synthesis on the reaction of semicarbazone creation. The peptides obtained had the ability to block the function of trypsin and other proteolytic components, but they also had a protective effect against the activity of pancreatotoxic substances caused by severe pancreatitis in animals. The shortfall of the patent proposed is the fully synthetic nature of the proposed substances; also, they do not have an absolute specificity to trypsin or chymotrypsin only; they react with the majority of macromolecules in the body due to the presence of semicarboxyl groups; they block the function of many enzymes, including those needed by the body to regain health. The fully synthetic nature of the product being patented creates the danger of many side effects: accumulation, absence of metabolism or the creation of toxic metabolites and carcinogenic and genotoxic properties. All these side effects are characteristic of semicarboxyls, and many of these derivatives are taken as mitotics for the treatment of cancer, which indicates their extreme toxicity. It is due specifically to this group that the reaction between the peptides being patented and the enzymes takes place very quickly, which does not allow the drug to accumulate selectively on the inflammatory threshold in cases of severe pancreatitis. The drug was insufficiently effective in the face of pancreatitis that had already developed and had only protective properties. The method for synthesizing semcarbazons has many stages and is expensive.

### DISCLOSURE OF THE INVENTION

At the heart of the invention is the task of developing a supramolecular ensemble of modified oligopeptides for the treatment of pancreatitis, stomach ulcers, and other hyperenzymemias based on enzyme peptide inhibitors and methods for obtaining them. The formula of modified oligopeptides must be quickly metabolized by the body without the creation of toxic metabolites, must inhibit only one enzyme and not affect the normal function of other enzymatic systems, must not be xenobiotic, must freely aggregate in the inflamed areas of the pancreas, and must penetrate not only the intercellular fluid, but also into the cells. We wish to quickly stop the inflammatory process caused by excess secretions of proteolytic enzymes. The technology of the proposed drug should be simple, economically accessible, ecologically harmless, and waste-free, and contain three stages.

The task set is addressed through the creation of formulae of modified oligopeptides for the treatment of pancreatitis, stomach ulcers, and other hyperenzymemias that have the capacity to inhibit proteolytic enzymes, where in the capacity of the main active ingredient, a mixture of chemically modified oligopeptides is used that are products of autological hydrolysis over the course of 0.2-48 hours of the proteolytic enzymes, with changes of their molecular charges to the opposite. For this purpose, the enzymes of trypsin, chymotrypsin, papain, pancreatin, or a mixture of enzymes is made into a solution and left for autolysis for 0.2-48 hours. Then the oligopeptides obtained have their structure modified, with a change in molecular charge to the opposite through acylation with succinic anhydride or alkylation with monochloracetic acid in a 10-200% relationship with the reagents by modifier mass to the mass taken in the protein reaction. Due to the effect of selective complementary hybridization, the peptides achieved interact only with their enzyme ancestors, thereby inhibiting their function. The low molecular weight of the peptides obtained and their immunity to hydrolytic enzymes allows them to easily penetrate through biological membranes, be absorbed from the intestine, and collect in areas with high concentrations of enzyme targets. These properties permit the peptides being patented to be used to obtain, among other things, peroral medicinal formulae for the treatment of chronic pancreatitis and stomach ulcers.

We used an assembly of oligopeptides that were the product of the autological hydrolysis of enzymes, but the molecules' charges were changed to the opposite. “Assembly” is a term from supramolecular chemistry. The objects of supramolecular chemistry are supramolecular assemblies that self-assemble out of their complements—that is, fragments that have geometrical and chemical correspondence—similar to the self-assembly of the most complex three-dimensional structures in a live cell [²³,²⁴]

### Best Invention Implementation Option

### EXAMPLE 1

### Obtaining a Trypsin Inhibitor

1.0 g trypsin is dissolved in 100 ml distilled water neutralized to pH=7.5 for the creation of a 1% solution; this is left to set for 48 hours at a temperature of 37° C. for autolysis. Then 2.0 g succinic anhydride is added to the peptide mixture obtained; this is stirred until fully dissolved. The solution of peptides obtained is poured into 5 ml test tubes, lyophilized, and used as a trypsin inhibitor.

### EXAMPLE 2

### A study of the Anti-Trypsin Activity of the Peptides Obtained

To determine the minimum effective concentration of the drug, a twofold dilution was prepared. An effective concentration is a dose of the peptide formula that fully inhibits the trypsin's proteolytic activity. In the capacity of a protein target for the action of the trypsin, 1% sodium caseinate with a phosphate buffer at a pH of 8.0. The concentration of oligopeptides in solution that were products of hydrolysis over time was determined by a spectrophotometer at 280 nm and 260 nm. A trypsin solution was added to a solution of 1% casein at an enzyme:protein ratio of 1:100; every five minutes, 1 ml of sample was taken and the same volume of 1% trichloracetic acid was added. The protein sediment created was centrifuged, and the concentration of dissolved peptides created after hydrolysis was determined by a spectrophotometer. The spectrophotometry method of protein determination is based on the ability of aromatic amino acids (tryptophan, tyrosine, and to a lesser extent, phenylalanine) to absorb ultraviolet light, with the maximum absorption at 280 nm. It is conditionally acceptable to believe that at a protein concentration in the solution equal to 1 mg/ml, the optical density value at 280 nm is equal to 1 when cuvettes with a layer thickness of 10 mm are used. The drug's eluent was used in the capacity of a comparison solution. The concentration of the experimental protein in the solution must be from 0.05 to 2 mg/l. The presence of nucleic acids and nucleotides (more than 20%) inhibit the identification of the protein. In this case, the optical density of the same solution is measured at two wavelengths: 260 and 280 nm; the amount of protein X (mg/ml) is calculated using the Calcar formula:

X=1.45·D₂₈₀−0.74·D₂₆₀.
The more dissolved peptides there were in the solution, the more active the trypsin was. Inhibiting trypsin should have led to decreasing the concentration of the dissolved peptides. The results of the study of the anti-trypsin activity of the peptide formula being patented follow.

TABLE 1
Dependence of Trypsin Activity on the Dilution
of Added Succinylpeptide Inhibitors

| | | | Concentration of |
|---|---|---|---|
| | | | Dissolved Peptides |
| | | | after One Hour of |
| | | Enzyme Activity | Incubation |
| No. | Specimen | (U/ml)* | (mg/ml)* |
| 1 | Trypsin (control) | 0.6 ± 0.05 U/ml | 10 ± 1 |
| 2 | Trypsin + | 0.1 ± 0.05 U/ml | 1.7 ± 0.2 |
| | succinylpeptides in a | | |
| | concentration of 0.125 | | |
| | ng/ml | | |
| 3 | Trypsin + | 0 | 0 |
| | succinylpeptides in a | | |
| | concentration of 0.25 | | |
| | ng/ml | | |
| 4 | Trypsin + | 0 | 0 |
| | succinylpeptides in a | | |
| | concentration of 0.5 | | |
| | ng/ml | | |
| 5 | Trypsin + | 0 | 0 |
| | succinylpeptides in a | | |
| | concentration of 1 | | |
| | ng/ml | | |

*P < 0.01

As may be seen in the table, the effective concentration of succinylpeptides is 0.125 ng/ml at a trypsin concentration of 0.1 mg/ml. The experiment also confirmed the effectiveness and dosage-dependent nature of the formula being applied.

### EXAMPLE 3

### Study of the Effectiveness of the Composition of Peptides in Treating Animals with Severe Pancreatitis

In the study, a model of severe pancreatitis in mice induced by interperitoneal introduction of caerulein was used [²⁵]. The intensity of the pancreatitis was correlated with the concentration of amylase in the blood of the mice. Pancreatitis was induced in mice 16-20 g in weight through intraparenteral introduction of caerulein in a single dose of 100 mcg/kg body weight. Caerulein was introduced again at an interval of six hours. To verify the hypothesis on the drug's effectiveness specifically in the treatment of pancreatitis, the formula of peptides obtained were introduced into the animals interparenterally at a dosage of 0.1 ml 1 ng/ml once a day for three days in a row. The concentration of amylase in the animals' blood was verified daily.

TABLE 2
Indicators of the Effectiveness of the Peptide Formula
Being Patented on Pancreatitis Models in Mice

| | Amylase | Number of Mice | Number of Mice |
|---|---|---|---|
| | Concentration | in Experimental | Dead within |
| Specimen | U/ml | Group | 10 Days |
| Caerulein and | 66.4 ± 11.1* | 12 | 6 |
| Physical Solution | | | |
| Caerulein and | 14.1 ± 2.3* | 12 | 0 |
| then the | | | |
| Formula Being | | | |
| Patented | | | |
| Control | 9.2 ± 1.1 | 5 | 0 |
| without | | | |
| Caerulein | | | |

*P < 0.01

As may be seen in the data presented in Table 2, the formula being patented turned out to be capable not only of normalizing the level of amylase in the blood of the animals nearly to the level of that of the control group, but also of preventing their deaths. While there was a 50% mortality rate in the control group, all the animals in the experimental group survived. Thus the formula of peptides being patented had a therapeutic effect on models of severe pancreatitis in mice.

### INDUSTRIAL APPLICABILITY

The technology of the production of this drug consists of three simple stages; the raw material for its production is commercially produced trypsin; no stage of production requires new, unique equipment or unique reagents. This invention may be quickly set up on the existing production lines and standardized unified equipment of pharmaceutical companies.

### REFERENCES

1 Enzymes as drugs/Ed. J. S. Holcenberg, J. Roberts. N.Y. etc.: Wiley and sons, 1981. 455 p.
2 Enzyme therapy in genetic diseases/Ed. R. J. Desnick. N.Y.: Alan Liss, 1980. Vol. 2. 450 p
3 Wiseman A. Enzymes in therapy—theory and practice.—In: Topics in enzyme and fermentation biotechnology. N.Y. etc., 1980, vol. 4, p. 9-24.
4 Enzyme therapy of lysosomal storage diseases/Ed. J. M. Tager et al. Amsterdam: North-Holland, 1974
5 Barlow G. H. Enzymes of clot lysis.≧In: Methods in enzymology. N.Y. etc., 1976, vol. 45, p. 239-280.
6 Vogel R., Trautschold L, Werle E. Natural proteinase inhibitors. N.Y.: Acad. press, 1958. 112 p.
7 Fritz H. Proteinase inhibitors in severe inflammatory processes (septic shock and experimental endotoxaemia): Biochemical, pathophysiological and therapeutic aspects.—In-Protein degradation in health and disease. Amsterdam: Excerpta Medica, 1980, p. 351-379.
8 Torchilin V. P., Martinek K. Enzyme stabilization without carriers.—Enzyme Microbiol. Technol., 1979, 1, p. 74-82.
9 Torchilin V. P. Immobilized enzymes and the use of immobilization principles for drug targeting.—In: Targeted drugs. N.Y. etc., 1983, p. 127-152.
10 Marshall J. J., Rabinowttz M. L. Enzyme stabilization by covalent attachment of carbohydrate.—Arch. Biochem. and Biophys., 1975, 167, p. 777-779.
11 Torchilin V. P., Il'ina E. V., Streltsova Z. A. et al. Enzyme immobilization on heparin.—J. Biomed. Mater. Res., 1978, 12, p. 585-590.
12 Kopecek J. Biodegradation of polymers for biomedical use.—In: IUPAC Macromolecules. Oxford; New York, 1982. 320 p.
13 Abuchowski A., McCoy I R, Palczuk N. C. et al Effect of covalent attachment of polyethylene glycol on immunogenicity and circulating life of bovine liver catalase.—J. Biol. Chem., 1977, 252, p. 3582-3586.
14 Newmark A., Abuchowski A. Murano G. Preparation an properties of adducts of streptokinase and streptokinase-plasmin complex with polyethylene glycol and pluronic polyol F38.—J Appl Biochem., 1982, 4, p. 185-189.
15 Geiger B., Specht B. U. von, Arnon R. Stabilization of human p-D-N-Acetylhexosaminidase A towards proteolytic inactivation by coupling it to poly(N-vinylpyrrolidone).—Europ. J. Biochem., 1977, 73, p. 141-147.
16 Sherwood R. F., Baird J. K., Atkinson T. et al. Enhanced plasma persistence of therapeutic enzymes by coupling to soluble dextran.—Biochem. J., 1977, 164, p. 461-464.
17 Liposomes in biological systems/Ed. G. Gregoriadis, A. C. Allison. N.Y.: Wiley and sons, 1980. 360 p.
18 Ang E., Glew R. Ihler G. Enzyme loading of nucleated chicken erythrocytes.—Exp. Cell Res., 1977, 104, p. 430-434.
19 Dale G. L., Beutler E. Enzyme replacement therapy in Gaucher's disease: A rapid, high-yield method for purification of glucocerebrosidase.—Proc. Nat Acad. Sci. US, 1976, 73, p. 4672-4674.
20 Hanger R., Folkman J. Polymers for the sustained release of proteins.—Nature, 1976, 263, p. 797-799.
21 Chazov E. L, Mazaev A. V., Lebedev B. S. et al. Experimental study of biosoluble drugs.—Thrombus lysis with biosoluble immobilized fibrinolysin in experiment.—Thrombosis Res., 1978, 12, p. 809-816.
22 U.S. Pat. No. 5,714,580 Trypsin inhibitors//T. Brunck, M. Pepe, D. Pearson, T. Webb
23 http://dic.academic.ru/dic.nsf/ruwiki/79240
24 Jean-Marie Lehn. Supramolecular Chemistry. Concepts and Perspectives.—Weinheim; New York; Basel; Cambridge; Tokyo: V C H Verlagsgesellschaft mbH, 1995.-P. 103 (Chapter 7)
25 Niederau C, Ferrell L D, Grendell J H. Caerulein-induced acute necrotizing pancreatitis in mice: protective effects of proglumide, benzotript, and secretin. Gastroenterology. 1985 May;88(5 Pt 1):1192-204.

1. Modified oligopeptides for the treatment of pancreatitis, stomach ulcers, and other hyperenzymemias based on an enzyme peptide inhibitor, distinct in that in the capacity of the main active ingredient, a mixture (assembly) of chemically modified oligopeptides are used that are products of autological hydrolysis of enzymes, with changes of their molecular charges to the opposite.

2. Modified oligopeptides for the treatment of pancreatitis, stomach ulcers, and other hyperenzymemias based on an enzyme peptide inhibitor according to claim 1, where trypsin is used as the enzyme.

3. Modified oligopeptides for the treatment of pancreatitis, stomach ulcers, and other hyperenzymemias based on an enzyme peptide inhibitor according to claim 1, where chymotrypsin is used as the enzyme.

4. Modified oligopeptides for the treatment of pancreatitis, stomach ulcers, and other hyperenzymemias based on an enzyme peptide inhibitor according to claim 1, where papain is used as the enzyme.

5. Modified oligopeptides for the treatment of pancreatitis, stomach ulcers, and other hyperenzymemias based on an enzyme peptide inhibitor according to claim 1, where a mixture of trypsin, chymotrypsin, and papain in any proportion is used as the enzyme.

6. Modified oligopeptides for the treatment of pancreatitis, stomach ulcers, and other hyperenzymemias based on an enzyme peptide inhibitor according to claim 1, where autolyzed pancreatin of animal origin is used as the enzyme.

7. Modified oligopeptides for the treatment of pancreatitis, stomach ulcers, and other hyperenzymemias based on an enzyme peptide inhibitor according to claim 1, where autolyzed pancreatin of genetically engineered origin is used as the enzyme.

8. Modified oligopeptides for the treatment of pancreatitis, stomach ulcers, and other hyperenzymemias based on an enzyme peptide inhibitor according to any one of claims 2-7, distinct in that the molecular charges of the oligopeptides that are the products of the enzyme autolysis are changed through over-acylation with succinic anhydride.

9. Modified oligopeptides for the treatment of pancreatitis, stomach ulcers, and other hyperenzymemias based on an enzyme peptide inhibitor according to any one of claims 2-7, distinct in that the molecular charges of the oligopeptides that are the products of the enzyme autolysis are changed through over-alkylation with monochloracetic acid.

10. Modified oligopeptides for the treatment of pancreatitis, stomach ulcers, and other hyperenzymemias based on an enzyme peptide inhibitor according to any one of claims 8-9, distinct in that the molecular charges of the oligopeptides that are the products of the enzyme autolysis are changed through over-modification at a level of modification equal to 10-200% of the mass of the protein taken in the reaction.

11. A method of obtaining modified oligopeptides for the treatment of pancreatitis, stomach ulcers, and other hyperenzymemias based on an enzyme peptide inhibitor, distinct in that enzymes are left for autolysis for 0.2-48 hours; then the structure of the oligopeptides obtained are chemically modified so that their molecular charges are changed to the opposite.

12. A method of obtaining modified oligopeptides for the treatment of pancreatitis, stomach ulcers, and other hyperenzymemias based on an enzyme peptide inhibitor according to claim 11, where trypsin is used as the enzyme.

13. A method of obtaining modified oligopeptides for the treatment of pancreatitis, stomach ulcers, and other hyperenzymemias based on an enzyme peptide inhibitor according to claim 11, where chymotrypsin is used as the enzyme.

14. A method of obtaining modified oligopeptides for the treatment of pancreatitis, stomach ulcers, and other hyperenzymemias based on an enzyme peptide inhibitor according to claim 11, where papain is used as the enzyme.

15. A method of obtaining modified oligopeptides for the treatment of pancreatitis, stomach ulcers, and other hyperenzymemias based on an enzyme peptide inhibitor according to claim 11, where a mixture of trypsin, chymotrypsin, and papain in any proportion is used as the enzyme.

16. A method of obtaining modified oligopeptides for the treatment of pancreatitis, stomach ulcers, and other hyperenzymemias based on an enzyme peptide inhibitor according to claim 11, where autolyzed pancreatin of animal origin is used as the enzyme.

17. A method of obtaining modified oligopeptides for the treatment of pancreatitis, stomach ulcers, and other hyperenzymemias based on an enzyme peptide inhibitor according to claim 11, where autolyzed pancreatin of genetically engineered origin is used as the enzyme.

18. A method of obtaining modified oligopeptides for the treatment of pancreatitis, stomach ulcers, and other hyperenzymemias based on an enzyme peptide inhibitor according to any one of claims 12-17, distinct in that the molecular charges of the oligopeptides that are the products of the enzyme autolysis are changed through over-acylation with succinic anhydride.

19. A method of obtaining modified oligopeptides for the treatment of pancreatitis, stomach ulcers, and other hyperenzymemias based on an enzyme peptide inhibitor according to any one of claims 12-17, distinct in that the molecular charges of the oligopeptides that are the products of the enzyme autolysis are changed through over-alkylation with monochloracetic acid.

20. A method of obtaining modified oligopeptides for the treatment of pancreatitis, stomach ulcers, and other hyperenzymemias based on an enzyme peptide inhibitor according to any one of claims 12-17, distinct in that the molecular charges of the oligopeptides that are the products of the enzyme autolysis are changed through over-modification at a level of modification equal to 10-200% of the mass of the protein taken in the reaction.
